SEQUENCE LISTING (2 sequences)

SEQ ID NO: 1 - protein, 6 aa, Artificial Sequence
  Linker peptide sequence
PLGLAG

SEQ ID NO: 2 - protein, 5 aa, Artificial Sequence
  Dendrimer peptide sequence
PAMAM

(19) United States
(12) Patent Application Publication
(10) Pub. No.: US 2016/0220687 A1
(43) Pub. Date: Aug. 4, 2016

(54) METALLOPROTEINASE-CLEAVABLE ALPHA-AMANITIN-DENDRIMER CONJUGATES AND METHOD OF TREATING CANCER

(71) Applicants: National Guard Health Affairs, Riyadh (SA); King Saud bin Abdulaziz University for Health Sciences, Riyadh (SA); King Abdullah International Medical Research Center, Riyadh (SA)

(72) Inventor: Omar Abdulaziz H. ALHAMDAN, Riyadh (SA)

(73) Assignees: National Guard Health Affairs, Riyadh (SA); King Saud bin Abdulaziz University for Health Sciences, Riyadh (SA); King Abdullah International Medical Research Center, Riyadh (SA)

(21) Appl. No.: 15/011,169

(22) Filed: Jan. 29, 2016

Related U.S. Application Data
(60) Provisional application No. 62/111,045, filed on Feb. 2, 2015.

(51) Int. Cl. A61K 47/48; A61K 38/12

(52) U.S. Cl. CPC A61K 47/48169; A61K 38/12

(57) ABSTRACT

A conjugate comprising a dendritic polymer or other scaffold, a metalloproteinase-cleavable linker and alpha-amanitin. Methods of using this conjugate for safe targeted treatment of cancer cells expressing metalloproteinases.

### CROSS-REFERENCE TO RELATED APPLICATIONS

This application claims the benefit of U.S. Provisional Application 62/111,045, filed Feb. 2, 2015 which is incorporated by reference in its entirety.

### BACKGROUND

1. Field of the Invention

The present invention relates to the fields of medicine and pharmacology especially to the targeted treatment of cancer cells that over-express metalloproteases compared to non-malignant cells. Targeted treatment is attained by administering a conjugate comprising a dendritic polymer, metalloproteinase-cleavable linker and alpha-amanitin. Surprisingly, this conjugate, cleaved by a MMP produced by a tumor cells, selectively targets highly toxic alpha-amanitin to the tumor cells and inhibits or reverses tumor growth.

2. Description of the Related Art

Matrix metalloproteinases (MMPs) are a large family of calcium-dependent zinc-containing endopeptidases which are involved in tissue remodeling and degradation of the extracellular matrix (ECM) which includes collagens, elastins, gelatin, matrix glycoproteins, and proteoglycans; Verma, et al., Bioorganic & Medicinal Chemistry 15 (2007) 2223-2268.

MMPs have been shown to be over-expressed or elevated in several types of cancer. MMPs 2 and 9 are elevated or expressed in prostate (10), SCC and BCC (1, 32), uterine cancer (17), breast cancer (4, 5, 7, 8, 20, 21, 27), ovary cancer (4), colorectal cancer (6), melanoma (3), gastric cancer (14), hepatocellular cancer (16), pancreatic cancer (16), mammary cancer (32, 12), glioblastoma (21), and lymphoma (18).

MMP2 expression by malignant prostatic epithelial cells is an independent predictor of decreased prostate cancer disease-free survival. In prostate cancer cases, stromal cell expressed MMP2 (75.9% of cases) and prostate cancer cells (70% cases). The inventors observed the highest levels of MMP2 expression in malignant prostate glands and in stromal fibroblasts. However, MMP2 expression was also noted in normal prostatic epithelial cells as well as in prostatic and vascular smooth muscle, albeit at a much lower level. A higher amount of MMP2 was associated with higher recurrence of prostate cancer. Expression of MMP2>50% of cell is major and independent predictor of decrease prostate cancer disease-free survival, and significant predictor of prostate cancer recurrence. (10)

In highly invasive human squamous carcinoma cell, TGF-β induced EMT (Epithelial-mesenchymal transition) leads to the upregulation of MMP2 through a snail (Zinc finger protein SNAI1) and an Ets 1 transcription factor dependent mechanism. The expression of MMP9 can be stimulated by SNAI1 through a MAPK and PI3K-dependent mechanism and was significantly associated with poor prognosis in early stage in oral squamous cell carcinoma (“OSCC”). (32)

Infiltration by mast cell and activation of MMP9 coincides with the angiogenic switch in pre malignant lesions during squamous epithelial carcinogenesis. Mice lacking MMP9 show reduced keratinocyte hyper-proliferation and decreased incidence of invasive tumors during skin carcinogenesis. MMP9 has been reported in dyskeratotic foci of Bowen's disease (SCC in situ) and in the infiltrating edges of micro invasive carcinomas. MT1-MMP expression was found in stromal fibroblasts surrounding the tumor islands in BCCs. Significantly higher level of TIMP2 (inhibitor of MMP2) were detected in less infiltrative BCC when compared with SCCs or infiltrative BCCs.

MMP2 correlated with the invasiveness of oral SCCs and shorter disease-free survival after treatment.

MMP9 correlated with angiogenic markers and poor survival in HNSCC patients.

MMP7, MMP9, and MT1-MMP, as well as gelatinase activity, were detected in carcinoma cells and correlated with depth of invasion. Expression of MMP7, MMP9, MMP 13, and MT 1-MMP has been detected in malignantly transformed keratocytes in SCCs (squamous cell carcinomas).

MMP2 and MMP9 are upregulated in invasive SCCs of skin as compared with BCCs (basal cell carcinomas). MT1-MMP positive cancer cells were detected especially at invasive edge of tumors. Collagen VII and IV were less expressed in SCC than BCC. Thus, reduction in collagen IV (major component of BM) combined with increase expression of both MMP2 and MMP 9 could account for the increase invasive potential of SCC vs. BCC. Beta 1, 3 integrins were prominently expressed at periphery of BCC tumor islands while they were absent or variably expressed in SCCs. (1)

MMP2 and MMP9 have been correlated with the processes of tumor cell invasion and metastasis in human cancer, including uterine neoplasms. Activity of MMP9 tends to increase from normal cervix to high grade intraepithelial lesion and SCC, and more advanced staged. Human papilloma virus (HPV) E6 and E7 gene transcription correlates with MMPs and TIMPs transcription. (17)

Galectin 1 expression, which promotes tumor invasiveness through upregulation of MMP 2 and MMP 9, was preferentially higher in more invasive parts of glioblastoma xenografts. (18). The Angiopoietin 2 (Ang2), MMP2, MT1-MMP, and Laminin (LN) 5 gamma 2 were co expressed in invasive areas but not in the central regions of glioma tissues. Ang2 induce the expression MT1-MMP (which is known to activate MMP2) and LN 5 gamma 2 in invasive glioma xenografts formed by human U87MG Ang2-expressing glioma cells in the murine brain and in cell culture. MT1-MMP binds to and activates proMMP2. Both MMP2 and MT1-MMP cleave LN 5 gamma2 and generate proteolytic fragments (gamma2′ and gamma2′X). The release of gamma2′ X is capable of promoting tumor cell invasion and preventing tumor cell apoptosis through interacting with the EGFR. (21)

Gal7 promotes T cell lymphoma progression through extracellular stimulation of MMP9 transcription. (18)

Breast cancer is the most common cancer affecting women in the world. Distant metastases are the principle cause of death. The cleavage of ECM by activated MMPs facilitates the invasion of tumor cells as well as the release of ECM bound growth factors (insulin like growth factors and fibroblast growth factors). Increased expression and activity of MMP2 and MMP9 in tumors leads to degradation of the basement membranes, an essential step in tumor invasion. High expression of MMP2 is associated with reduced survival in breast cancer patients Increased levels of MMP9 in breast cancer tissue is associated with tumor grade. Treatment of MCF7 breast cancer cells with Letrozole (aromatase-inhibitor) lead to efficient reduction of MMP2 and MMP9 levels that suppresses both breast cancer growth and invasion. Higher expression of MMP9 and 11 correlated with higher rate of distant metastases. Higher expression of MMP2, MMP7, MMP9 and MMP11 mRNA is found in breast cancer tissue than in normal breast tissue. Significant differences were observed between normal breast tissue and grade 2 breast cancer tissue concerning the mRNA levels for MMP9 (p=0.0127), MMP11 (p 0.0007), and MMP12 (0.008). MMP2 was found in the cytoplasm of normal breast and endothelial cells while MMP2 only stained the nuclei of tumor cells (5).

MMP2 and MMP14 deficient mice display diminished ductal elongation. Reduction of TIMP 1 expression lead to more extensive branching, increase ductal elongation, and increase proliferation index. TIMP3 deficient mice show accelerated ductal elongation but normal branching patterns. Over-expression of MMP14 in mammary epithelium leads to increased lymphatic infiltration, peri ductal fibrosis, ductal hyperplasia and dilated ducts, dysphasia, and adenocarcinoma in multiparous transgenic mice. High levels of MMP9, which degrade collagen IV in basement membrane, are associated with poor prognosis in breast cancer independent of cell type. In primary invasive ductal carcinoma study, high individual expression of MMP9, MMP11, TIMP1, and TIMP2 were significantly associated with increased incidence of metastasis at 5 year postsurgical resection. In mammary tumorigenesis xenograft model, CD4⁺ T cells in periphery as well as within the breast tumor expressed high levels ofMMP9 (8).

MMP1 and MMP2 have been described as genes that selectively mediate lung metastasis in breast cancer mouse model. Peri tumoral fibroblasts are the main producers of MMP1, MMP2, MMP3, MMP11, MMP13, and MMP14 in breast cancer. In invasive breast cancer, mRNA and membranous MT1-MMP (but not cytoplasmic protein expression) has been correlated with Lymph node metastasis. Expression of MMP3 and MT1-MMP (activator of MMP2) is sufficient to stimulate the development if invasive tumors. MMP 2, MMP3, MMP11, MT1-MMP, MT4-MMP are positive regulators of progression (cancer promotors).

MMP8 negatively regulates metastasis in breast cancer models (20). LN5 gamma 2 and MT1-MMP have been found to be co localized in invasive fronts of human breast cancer (21). MMP2 mRNA was detected in 8/8 patients investigated. PyMT tumors were uniformly distributed throughout the stroma in both interior and periphery of the tumors.

TGF-β is expressed by cancer cells in breast cancer, which can induce differentiation of normal breast fibroblasts to myofibroblasts (characteristic stromal cell type in many carcinomas. The blockage of TGF-β signaling in FVB-PyMT mice has been shown to reduce MMP2 and MMP9 activity and to decrease lung metastasis (27).

MMP2 is associated with integrin alpha v beta 3 and this interaction is essential for localizing the enzyme to the surface of newly formed vessels. MMP2 null mice showed reduced vascularization compared to wild type. MMP2/MMP9 double deficient mice have shown severe impairments in choroidal neovascularization. Mice deficient in MMP 2, MMP3, or MMP9 have a lower level of apoptosis induced by TNF-alpha. MMP9 induced in tumor macrophage and endothelial cells and promote lung metastasis (4).

Relaxin induces increases in MMP 2, MMP9, and MMP7 and up regulation of mRNA of MMP2, MMP9, MMP13, and MMP14 which cause increased tumor migration of invasive breast cancer cell line, especially with estrogen-independent cells. This effect is abolished by MMPI (NF439) to control level. Relaxin has been known predominantly for its effects on reproductive system, where it induces remodeling of ECM and upregulation of MMPs. Relaxin decrease the synthesis of collagen and increase the expression and activities of MMP 1, MMP2, MMP9, and MMP3. Exposure to relaxin was followed by significantly increased invasiveness of tumor cells on basis of increase production of MMP 2, MMP7, MMP9, MMP13, and MMP14. The predominant form was MMP 9 (active form), while MMP2 was detectable mostly in latent form. Concomitant upregulation of MMP 2 and MMP 14 has been shown to increase activation of proMMP2 and to be associated with lymph node and distant metastases (7). In the aggressive form of this tumor, increased expression and activity of proMMP9, but not its active form, was shown (5). MMP9 contributes to the malignant behavior of ovarian cancer by promoting neovascularization (4).

There is progressive and significant increase of proMMP 9 activity from adenoma to colorectal carcinoma tissue. ProMMP9 was significantly higher in advanced versus nonadvanced adenomas and in those harboring high grade dysplasia. MMP 9 is markedly upregulated in adenomatous tissue. The activity of latent and active form of MMP 2 was exclusively upregulated in colorectal carcinoma samples. Enhanced MMP 9 expression in the tumor compared to normal tissue has been associated with increase disease recurrence and reduction in overall survival. The four flat-depressed lesions (which associated with higher malignancy risk than flat elevated or protruded adenomas) included despite their small size had upregulated proMMP9 and active MMP2. Activation of proMMP9 is early event in the colorectal adenoma-carcinoma sequence, whereas MMP2 activation seems to be a late phenomenon in colorectal carcinogenesis. In advanced CRC the expression of MMP2 and 9 has been associated with tumor invasion, hepatic metastasis, and decrease survival rate (6).

Over-expression of MMP2 in melanoma has been shown by a number of preclinical and clinical investigations. MMP2 is highly expressed in human melanomas and its expression is correlated with hematogeneous metastasis and prognosis in melanoma patients (3). TIMP1 (an inhibitor of MMP9) administration in mice injected with B16F10 melanoma cells reduced the number of pulmonary metastases. Over-expression of TIMP3 induces apoptosis in variety types of malignant cells (14).

Marimastat is an inhibitor of MMP1, MMP2, MMP3, MMP7, MMP9, MMP 12, and MMP 13. Marimastat treatment of patients with advanced gastric cancer demonstrated significant improvement in disease free survival and overall survival compared to treatment with a placebo (4, 14).

MT1-MMP is associated with poorer prognosis in hepatocellular carcinoma (HCC). In HCC, MT1-MMP mRNA is mainly expressed by cancer cells and to lesser extent in stromal cells. In HCC, MMP2 mRNA is expressed predominantly by tumor stroma. While MMP2 is present in cancer and stroma cells, its active form is only found in cancer cells. Increased amounts of latent and active MMP are found in tumor cells compared with non-tumorous liver tissue. In HCC, MMP 9 mRNA is found mainly in neoplastic epithelial cells. MMP9 content was found to be almost equal to that of normal liver tissue, but was active only in HCC. MT1-MMP was associated with lower degree of differentiation in HCC but not in pancreatic cancer (16).

In pancreatic cancer, MT1-MMP mRNA is moderately expressed in cancer and stromal cells and MT1-MMP equally expressed in both cancer and stroma. However, in pancreatic cancer, MMP 2 mRNA was predominantly expressed by tumor stroma. In pancreatic cancer, MMP 9 mRNA is moderately expressed in cancer and stromal cells (16).

Nexin1 binds low-density lipoprotein-receptor-related protein 1 (LRP1) and stimulates extracellular signal-regulated kinase signaling, MMP 9 expression, and metastatic spread of mammary tumors. Activating MMP9 and TGF-B signaling at the tumor-bone interface by MMP13 regulates mammary tumor-induced osteolysis, a common feature of a variety of cancers (32). T cells appear to be key contributors of MMP9 in the mammary tumor model (12).

Cells that express MMPs have been previously identified with special dyes that label tumor cells expressing MMPs. These methods use special fluorescent dyes to label tumor tissues during excision surgery. In order to avoid missing any part of the tumor and avoid to injuring adjacent nerves in the excision surgery the dyes are conjugated to an MMP cleavable compound. This MMP cleavable compound releases the fluorescent dye when contacted with MMPs released by tumor cells and thus is a means for selective colorization of tumors. Prior art conjugates are used to color-code tumor tissues as described by http://_www.ted.com/talks/quyen_nguyen_color_coded_surgery (last accessed Jan. 24, 2016).

Conjugates of dendritic polymers and cyclic peptides have been used to target tumor tissues. Olson, et al., Integr. Biol. 1:382-393 (2009) describes polycationic cell penetrating peptide connected via an MMP cleavable linker to a neutralizing polyanion. However, it reports that the cell-penetrating peptides accumulate more strongly in cartilage, liver and kidney than in tumors. Olson reports that attachment of cell penetrating peptides to macromolecular carriers, such as PAMAM dendrimers, reduces uptake by kidney and joint tissues probably due to the large size of the conjugates precluding them from passing through pores in kidney glomeruli and pores in cartilage. Alpha-amanitin has not been previously conjugated to dendritic polymers.

Alpha-amanitin inhibits RNA polymerase II, causing protein deficit and ultimately cell death. The liver is the main target organ of toxicity, but other organs are also affected, especially the kidneys. Intoxication symptoms usually appear after a latent period and may include gastrointestinal disorders followed by jaundice, seizures, and coma, culminating in death. Therapy for alpha amanitin toxicity consists in supportive measures, gastric decontamination, drug therapy and, ultimately, liver transplantation if clinical condition worsens. The discovery of an effective antidote is still a major unsolved issue. Garcia, et al., Food Chem Toxicol. 2015 Sep. 12; 86:41-55.

The intravenous or intraperitoneal administration of alpha-amanitin has causes serious pathological effects on the liver and kidney and it has been reported that the cyclopeptide alpha-amanitin could reside in these two organs for a long last toxic effect; Cao, et al., Sichuan Da Xue Xue Bao Yi Xue Ban. 40(5):901-4 (2009). When alpha-amanitin is conjugated to an antibody very little free amanitin was released. Parmley, S. SciBX 7(48); doi:10.1038/scibx.2014.1397 Published online Dec. 18, 2014. Hechler, et al., AACR Annual Meeting 2014, #664, “Amanitin-based antibody-drug conjugates targeting the prostate-specific membrane antigen PSMA “report conjugation of alpha-amanitin via a cleavable linker to antibodies that bind to prostate cancer cell surface receptors and release alpha-amanitin inside of the cell's lysosomes. However, these methods require the use of an antibody conjugate that selectively binds to cancer cells and once internalized releases alpha-amanitin inside of a cell's lysosomes.

Alpha-Amanitin inhibits the action of RNA polymerases, especially RNA polymerases 2 and 3; Vetter J., Toxicon. 36(1):13-24 (1998) which impairs cell functions and biological activities leading to cellular death. Amanitins are a class of toxic compounds produced by particular fungi. They include alpha-, beta- and epsilon-amanitin. Alpha-amanitin is one type of thermostable cyclic oligopeptide toxin contained naturally in Amanita phalloides mushrooms. Alpha-amanitin or α-amanitin is a cyclic peptide of eight amino acids:

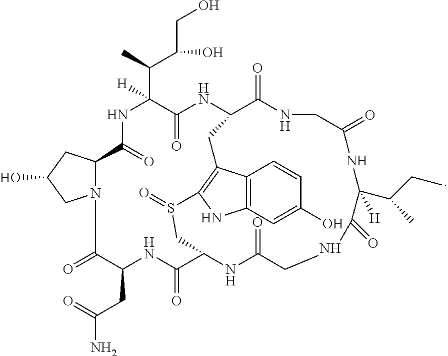 [SMILES: CC[C@H](C)[C@@H]1NC(=O)CNC(=O)[C@@H]2Cc3c([nH]c4cc(O)ccc34)S(=O)C[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

α-Amanitin has an unusually strong and specific attraction to the enzyme RNA polymerase II. Upon ingestion and uptake by liver cells, it irreversibly binds to the RNA polymerase II enzyme, effectively causing cytolysis of hepatocytes (liver cells), Michelot, et al., Drug Metabol Drug Interact 6 (3-4): 265-274 (1988). Immunogenic conjugates of alpha-amanitin have been used to produce antibodies against it, Zhelev, et al., Toxicon, 25(9): 981-987 (1987).

In view of the need for a highly effective targeted therapy for cancer cells expressing MMPs, the inventor investigated whether the highly toxic alpha-amanitin could be used to selectively kill cancer or tumor cells without imposing a substantial toxic burden on a subject undergoing treatment.

In some embodiments the invention provides an anti-tumor treatment with double selectivity for tumor or cancer cells. Alpha-amanitin selectively kills growing tumor or cancer cells because it targets RNA polymerase necessary for the high protein synthesis activity in these rapidly growing cells. Second, a conjugate containing an MMP cleavable linker selectively releases alpha-amanitin in the presence of a MMP produced or over-expressed by a tumor or cancer cell.

### BRIEF SUMMARY OF THE INVENTION

The invention is directed to a conjugate of a dendritic polymer, an MMP cleavable linker, and α-amanitin. The MMP cleavable linker is cut by an MMP produced by a tumor or cancer cell, which releases α-amanitin, which is taken up by the tumor or cancer cell. The α-amanitin inhibits RNA polymerase(s) in the tumor or cancer cell thus inhibiting the RNA polymerase(s) in the cell. This leads to death or inhibition of the tumor or cancer cell.

Normal cells which do not express the high level of MMP of the targeted tumor or cancer cell, do not cut the cleavable linker of the conjugate and are not exposed to significant amounts of free or cleaved α-amanitin which remains attached to the large dendritic polymer.

In contrast, rapidly growing tumor cells need more proteins for their uncontrolled growth and expansion. They produce higher amounts of MMPs for their process of invasion and metastasis. The higher levels of MMPs produced by these tumor cells cleave the conjugate of the invention, release free or cleaved alpha-amanitin. The free alpha-amanitin targets RNA polymerase II locally and irreversibly disturbs the internal protein synthesis in the tumor cells leading to slowing of tumor growth and eventual death of the tumor cells.

The activity of the conjugate of the invention may occur through at least two different mechanisms. Tumor or cancer cells expressing high amounts of an MMP, such as MMP2 or MMP9, will be exposed to higher amounts of toxic alpha-amanitin because the higher amounts of MMP will more efficiently locally cleave alpha-amanitin from the conjugate. Second, tumor or cancer cells that express high amounts and/or activity of RNA polymerases needed to express proteins (e.g., in rapidly growing or proliferating tumor or cancer cells), present or contain more RNA polymerase targets for the locally released alpha-amanitin than normal cells expressing lower amounts of RNA polymerases.

### BRIEF DESCRIPTION OF THE DRAWINGS

FIG. 1A depicts a tumor cell releasing matrix metalloproteinases (“MMPs”) and a conjugate comprising a “killer” moiety such as an alpha-amanitin, a linker (MMP site of action) depicted by the vertical dotted line, and a carrier (neutralizing) moiety. The PLGLAG (SEQ ID NO: 1) sequence of amino acids is moderately selective to MMP2 and MMP9 (11). A representative killer moiety is alpha-amanitin. Representative neutralizing moieties are dendrimers of PAMAM (SEQ ID NO: 2)(11).

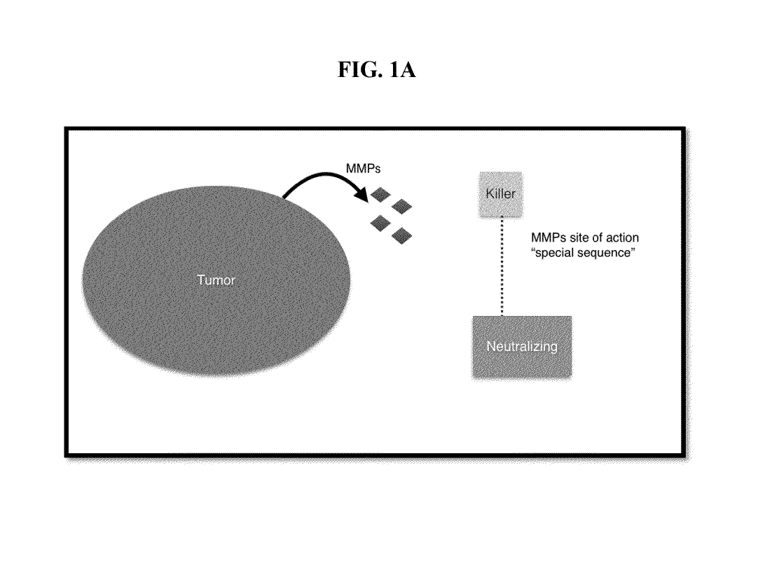

FIG. 1B depicts the selective cleavage of the linker by MMPs (diamonds) yielding released killer peptides (squares) that are taken up by tumor cells expressing MMPs. The MMP acts as the scissors that releases the cleavable toxic alpha amanitin moiety which is a patent RNA polymerase inhibitor. Interaction of the released alpha amanitin toxin with the tumor cell eventually kills it. While not being bound to any particular mechanism, the alpha-amanitin released by cleavage of the MMP cleavable linker may interact with the tumor cell after binding to its surface or after internalization, intracellular processing and/or further trafficking by the tumor cell.

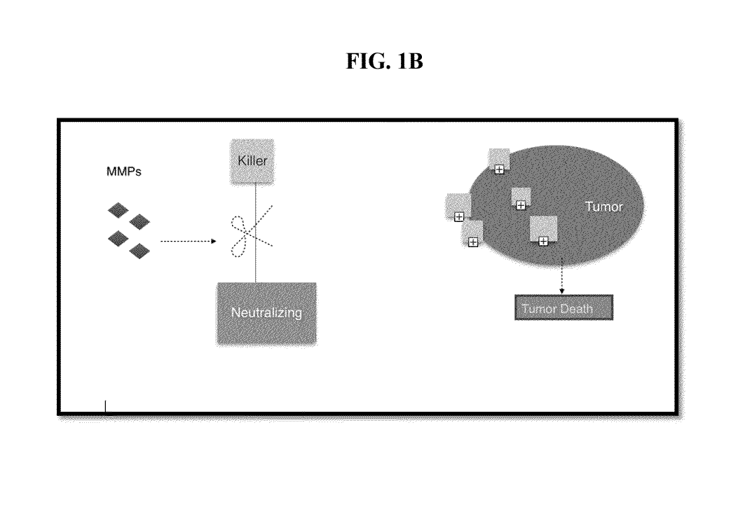

### DETAILED DESCRIPTION OF THE INVENTION

Aspects of the invention include compositions and methods for inhibiting the growth or for killing cancer, neoplastic or tumor cells that express more active matrix metalloproteinases than surrounding normal cells. These compositions and methods find a number of uses including suppressing tumor growth, tumor metastasis, and/or reducing tumor load, for example, by reducing tumor size or tumor number. These and other objects, advantages, and features of the invention will become apparent to those persons skilled in the art upon reading the details of the compositions and methods as more fully described below.

The alpha-amanitins according to the invention preferably have the core structure shown below.

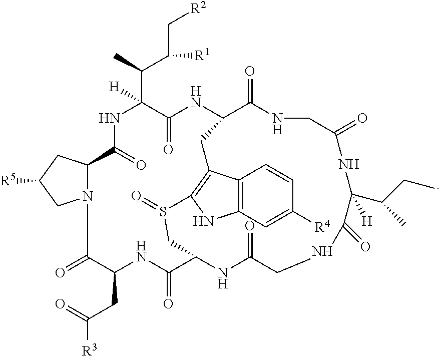

wherein R1, R2, R3, R4 and R5 are, independently, H, OH, or NH₂.

A derivative having the core structure shown modified at the R group positions may also be employed provided that they exhibit an ability to inhibit cancer or tumor cell growth, for example, by inhibiting RNAP II.

Chemical derivatives of amanitin, such as OMA-gly, OMA-pro, OMA-COOH, and OMA-NH, which may have a lower affinity for RNAP II may also be used to form the conjugates of the invention. Such chemical derivatives as well as methods for their production are described by Mullersman, Chemical Modification of Alpha-Amanitin to Yield Derivatives Suitable for Conjugation to Proteins via Reductive Alkylation (1986), which is incorporated by reference.

A releasable alpha-amanitin peptide is released from a larger conjugate by the action of a MMP on a linker or cleavable portion of the conjugate containing the releasable alpha amanitin moiety. When part of a conjugate the cleavable alpha-amanitin peptide moiety is bound via one or more matrix metalloproteinase-cleavable linker(s) to a carrier. More than one alpha amanitin peptide or moiety may be bound to a carrier via a cleavable linker or moiety and a carrier, such as a dendritic carrier may contain multiple moieties comprising alpha amanitin peptides and intervening cleavable linkers. Thus, a conjugate may contain a single releasable alpha-amanitin peptide or more than one. One or more alpha-amanitin peptide moieties may be linked to one or more linkers, for example, to modulate its release and pharmacodynamics properties, it may be configured so that it is released by a single, dual or multiple cleavage events.

In free or released form the released peptide has been detached from the conjugate by cleavage of the linker(s) by a matrix metalloproteinase, generally by an MMP that is up-regulated, over-expressed or aberrantly-expressed by a tumor or cancer cell compare to a reference, cell such as a normal control cell of the same type as the tumor or cancer cell.

The terms “up-regulated”, “over-expressed” and “aberrantly-expressed” are measured by comparison to a reference cell, such as to a normal, untransformed cell of the type from which the tumor or cancer cell developed or from a different type of normal cell associated with a cancer or tumor. In other embodiments, a reference cell may be another cancer or tumor cell, for example, one that expresses a lower or normal amount of an MMP.

Matrix metalloproteinases (MMPs) are a group of zinc dependent matrix degrading enzymes (30). Degradation of extracellular matrix by MMPs contributes to removal of physical barrier to cancer, which occurs at several stages of the tumorigenesis including local invasion, neoangiogenesis and extravasation , and being involved in ability of cancer to invade locally and distally metastasize (31).

The terms “subject,” “individual,” and “patient” are used interchangeably to refer to a mammal being assessed for treatment and/or being treated. In a preferred embodiment, the mammal is a human. The terms “subject,” “individual,” and “patient” thus encompass individuals having cancer (e.g., colorectal cancer, adenocarcinoma of the ovary or prostate, breast carcinoma, lung carcinoma, etc.), including those who have undergone or are candidates for resection (surgery) to remove cancerous tissue (e.g., cancerous colorectal tissue). As described above, a subject may be human, but also include other mammals, particularly those mammals useful as laboratory models for human disease, e.g. nude-, immunodeficient-, knock-out-mice, rats, etc.

As used herein, the terms “treatment,” “treating,” and the like, refer to administering an agent, or carrying out a procedure (e.g., chemotherapy, radiation, a surgical procedure, etc.) for the purpose of obtaining an effect. The effect may be prophylactic in terms of completely or partially preventing a disease or symptom thereof and/or may be therapeutic in terms of affecting a partial or complete cure for a disease and/or symptoms of the disease. “Treatment,” as used herein, covers any treatment of any non-metastatic or metastatic tumor in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. In tumor or cancer treatment, a therapeutic agent may reduce the rate in which a tumor size is increasing or new tumors are forming; it may prevent any increase in tumor size or numbers of tumors formed; it may directly decrease the size of tumors and/or metastasis of tumor cells; or may transform a cell toward a more normal phenotype.

“In combination with”, “combination therapy” and “combination products” refer, in certain embodiments, to the concurrent administration to a patient of the conjugates as described and one or more supplementary agents or treatments. When administered in combination, each component can be administered at the same time or sequentially in any order at different points in time. Thus, each component can be administered separately but sufficiently closely in time so as to provide the desired therapeutic effect.

According to the present invention, the supplementary agent(s) can be any suitable therapeutic agent, such as a drug, antibody, immunotherapeutic agent (e.g., T cell, B cell or other leukocyte), or other agent cytotoxic or inhibitory for tumor or cancer cells. Other combination therapies include radiation treatment, surgery, and hormone deprivation (see Kwon et al., Proc. Natl. Acad. Sci. U.S.A., 96: 15074-9, 1999 incorporated by reference).

Such supplemental agents may include cofactors, such as Zn or Ca, that activate MMPs or other agents that up-regulate MMP expression in cancer cells. A supplementary agent may also be an MMP chelator or inhibitor, such as hydroxamates, carboxylates, thiols, and phosphinyls or an antibody or other ligand that binds to an MMP. In some embodiments, cofactors, activators or inhibitors are used to modulate differential expression of MMPs by cancer and tumor cells compared to normal cells to attain a high degree of selective toxicity for the conjugates according to the invention.

“Concomitant administration” of a known cancer therapeutic drug with a pharmaceutical composition of the present invention means administration of the drug and a conjugate according to the invention at such time that both the known drug and the conjugate of the present invention will have a therapeutic effect. Such concomitant administration may involve concurrent (i.e. at the same time), prior, or subsequent administration of the drug with respect to the administration of a compound of the present invention. A person of ordinary skill in the art would have no difficulty determining the appropriate timing, sequence and dosages of administration for particular drugs and compositions of the present invention.

The term “cell culture” or “culture” means the maintenance of cells in an artificial, in vitro environment. It is to be understood, however, that the term “cell culture” is a generic term and may be used to encompass the cultivation not only of individual cells, but also of tissues or organs. In some embodiments, cells or tissues may be removed from a subject, viably maintained or cultured, treated ex vivo or in vitro with a conjugate according to the invention, and reintroduced into the subject.

The term “tumor,” as used herein, refers to all neoplastic cell growth and proliferation, whether malignant or benign, and all pre-cancerous and cancerous cells and tissues.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. In general, cells of interest for detection, analysis, classification, or treatment in the present application include precancerous (e.g., benign), malignant, pre-metastatic, metastatic, and non-metastatic cells. Examples of cancer include but are not limited to breast cancer, mammary cancer, colorectal cancer, hepatocellular cancer, gastric cancer, pancreatic cancer, ovarian cancer, prostate cancer, glioblastoma, lymphoma, squamous cell carcinoma (SCC), and basal cell carcinoma (BCC).

The term “cancer cell proliferation” refers to the proliferation of neoplastic cells that results in the growth of a tumor or increase in tumor load or tumor cell number.

The term “cancer metastasis” or “metastasis” refers to the spread of cancer cells from one organ or part of an organ to another, non-adjacent organ or organ part. In other words, it is the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor. Metastasis can also be defined as several steps of a process, such as the departure of cancer cells from an original tumor site, migration to another site, and invasion of cancer cells into other tissues of the body. Therefore, the present invention contemplates a method of treating, i.e. suppressing, preventing, or halting, growth of one or more cancerous tumors in an organ or body part which is not directly connected to the organ of the original cancerous tumor, and/or any steps in a process leading up to that growth.

Over-expression of MMPs is associated with the ability of a cancer to invade locally and distally metastasize. Proteases can favor tumor progression in multiple ways. Proteolytic degradation of the extracellular matrix favors tumor growth, local invasion, tumor cell extravasation, post-extravasation growth and survival as well as intravasation. Matrix-degrading enzymes are involved in the release of factors stored inside the matrix and in proteolytic activation of proteins, thus indirectly contributing to tumor progression. Matrix degradation in tumor-associated new vessel formation (angiogenesis) has been described. The targeted administration of the alpha-amanitin conjugate of the invention can reduce these phenomena by eliminating cancer or neoplastic cells over-expressing MMPs that degrade extracellular matrix and that contribute to removal of physical barriers to the spread of cancer.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after treatment of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis.

The term “diagnosis” is used herein to refer to the identification of a molecular or pathological state, disease or condition, such as the identification of a molecular subtype of breast cancer, prostate cancer, or other type of cancer. In some embodiments, a conjugate according to the invention, which contains both a releasable alpha-amanitin peptide and a MMP cleavable linker, may further comprise a label or other detectable marker. In addition to the therapeutic properties provided by the invention, such embodiments can be used to assess anti-tumor specificity and otherwise follow the course of therapy. In some embodiments diagnosis involves classifying, selecting or grouping subjects determined to have cancers that over-express or aberrantly-express MMPs and treating the subjects with a conjugate according to the invention. Diagnosis may also involve excluding subjects who have cancers that do not over-express MMPs from treatment with the conjugate according to the invention and treating them with alternative regimens.

The term “prognosis” is used herein to refer to the prediction of the likelihood of cancer, or death or progression attributable to cancer, including recurrence, metastatic spread, and drug resistance, of a neoplastic disease, such as lung, colon, skin or esophageal cancer. The term “prediction” is used herein to refer to the act of foretelling or estimating, based on observation, experience, or scientific reasoning. In one example, a physician may predict the likelihood that a patient will survive, following surgical removal of a primary tumor and/or chemotherapy for a certain period of time without cancer recurrence.

The term “isolated” is intended to mean that a compound is separated from all or some of the components that accompany it in nature. “Isolated” also refers to the state of a compound (e.g. protein) separated from all or some of the components that accompany it during manufacture (e.g., coproducts or contaminants resulting from chemical synthesis, recombinant expression, culture medium, and the like).

The term “specific binding” refers to that binding which occurs between such paired species as MMP and a linker cleavable by the MMP, an alpha-amanitin peptide and a cell surface, cellular receptor or other cell component, or other paired interactions such as between enzyme/substrate, receptor/ligand, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or ligand/receptor interaction or which initiates a functional interaction of the bound components, such as between an enzyme (e.g., MMP) and a site recognized by the enzyme (e.g., MMP-cleavable linker).

Aspects of the invention include compositions and methods for targeting cancer cells that express above normal amounts or concentrations of MMPs or above normal amounts of polymerases and/or more highly active polymerases. These compositions and methods find a number of uses including, for example, suppressing tumor growth and metastasis and reducing tumor size and tumor number in a mammal with cancer.

By “cancer cell proliferation”, it is meant the uncontrolled proliferation of cancer, tumor or other neoplastic cells that results in the growth or expansion of the cancer, tumor or neoplasm.

By “metastasis” it is meant the spread of cancer from one organ or tissue to a non-adjacent organ or tissue. Accordingly, methods of the invention find use in preventing the growth and spread of tumors.

By “suppressing” or “preventing” cancer cell proliferation and metastasis, i.e. the growth and spread of tumors, it is meant slowing the rate of proliferation and/or metastasis relative to the rate that would be observed prior to performing the methods of the invention, e.g. by 2-fold or more, 3-fold or more, 4-fold or more, including 5-fold or more, 7-fold or more, or 10-fold or more, such as 20-fold or more, or 50-fold or more, etc., relative to the rate of proliferation and/or metastasis prior to performing the methods. Proliferation and metastasis may be assessed by any convenient in vitro or in vivo method. For example, the rate of proliferation may be assessed in vitro by, e.g., counting the number of cells in culture that divide over a period of time with ³H-thymidine, while the rate of metastasis may be assessed by, e.g., the extent to which neoplastic, i.e. cancer, cells invade tissue preparations in organ cultures. As another example, proliferation may be assessed in vivo by, e.g., measuring tumor size over time, i.e. before treatment and at one or more time points after the start of treatment, e.g. 1 week, 2 weeks, 30 days, 60 days, and/or 90 days or more after the start of treatment, while the rate of metastasis may be assessed by, e.g., measuring the number of metastases observed in whole body imaging over time, before and after the start of treatment. In some instances, proliferation and/or metastasis may be slowed so substantially so as to be negligible, i.e. unmeasurable, i.e. halted. In other words, no tumor growth or new tumors may be observed. As demonstrated in the examples below, in some instances, tumor growth may be reversed, i.e. tumors may begin to shrink, e.g. by 2-fold, 3-fold, or 4-fold, or more, including 5-fold or more, 7-fold or more, 10-fold or more, in some cases 20-fold or more, 50-fold or more, etc., or the number of tumors may begin to decrease.

RNA Polymerase II synthesizes the precursors of mRNA that are subsequently translated to produce proteins. Polymerase II also synthesizes certain small nuclear RNAs, snRNA.

Non-limiting embodiments of the invention include the following.

A conjugate comprising a releasable cell alpha-amanitin peptide linked to at least one matrix metalloproteinase-cleavable linker to at least one carrier; wherein the cell-penetrating peptide is releasable from the conjugate when contacted with a matrix metalloproteinase that cleaves the metalloproteinase-cleavable linker. In this conjugate, the releasable alpha-amanitin peptide may comprise the following core structure:

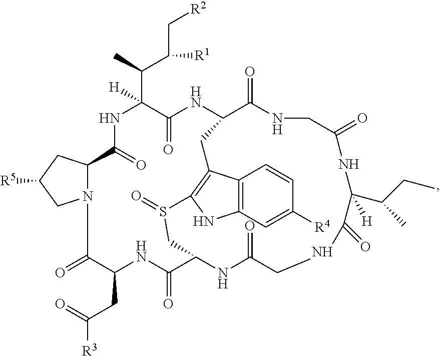

wherein R1, R2, R3, R4 and R5 are, independently, H, —R, OH, —OR, —NH₂, —NHR, —NRR, where R is an optionally substituted C₁-C₆ alkane, alkene or alkyne. In some embodiments the releasable peptide is alpha-amanitin, in others the releasable peptide is an alpha amanitin that is covalently-linked to a cleavable linker and comprises formula (I) linked via at least one atom to the cleavable linker. In some embodiments the releasable peptide may further comprise a detectable marker, such as a releasable cell-penetrating alpha amanitin peptide tagged with a detectable marker. Such a detectable marker may allow one to determine the amount of uptake of the releasable peptide by target cells and assess the efficacy and specificity of treatment.

Such a conjugate may contain a cleavable linker that is cleavable by a secreted matrix metalloproteinase, such as by a membrane-bound matrix metalloproteinase. The cleavable linker may be selectively cleavable by matrix metalloproteinase 2 or 9. The cleavable linker may comprise the amino acid sequence PLGLAG (SEQ ID NO: 1). Many substrate preferences for different MMPs are known, so that a cleavable linker can be designed that will be preferentially cleaved by a particular class or species of MMP. Such a linker may be selected to be preferentially bound by membrane-anchored MMPs localized to the outer surface of a tumor or cancer cell. Alternatively, a linker may be selected to be cleaved by a soluble secreted MMP that in vivo would be proximal to a tumor or cancer cell expressing the MMP. Suitable linkers that are cleavable by MMPs include those disclosed by Jiang, et al., U.S. Pat. No. 9,072,792 which is hereby incorporated by reference.

The conjugate according to the invention comprises a carrier. Such a carrier may be a dendrimer or other macromolecular carrier, such as a dendrimer comprising the amino acid sequence PAMAM (SEQ ID NO: 2). PAMAM (SEQ ID NO: 2) dendrimers and methods for making and using them are incorporated by reference to Esfand et al., “Poly(amidoamine) PAMAM (SEQ ID NO: 2) dendrimers: from biomimicry to drug delivery and biomedical applications,” DDT 6(8): 427-436 (2001). Other polymeric scaffolds suitable for administration in vivo are known in the artand can be selected to permit conjuation of a MMP cleavable linker and an alpha amanitin moiety. Suitable multivalent polymeric scaffolds, including other dendrimers, are known to those skilled in the art, and can be selected depending on the agents with which it is operatively associated as well as the desired properties of the scaffold.

A conjugate may further comprise a targeting moiety that binds to a tumor- or cancer-associated antigen. A targeting moiety may comprise an antibody, lectin or other compound that specifically or preferentially binds to antigens or other compounds expressed by tumor or cancer cells.

A conjugate according to the invention may be prepared of formulated as a composition containing a pharmaceutically acceptable excipient, such as a liquid excipient such as normal or buffered saline solution. Such a composition may further comprise at least one agent that modulates a pharmacokinetic or pharmacodynamic property of the matrix metalloproteinase, such as increasing the activity of a MMP expressed by a cancer cell or decreasing the activity of a MMP expressed by a normal cell.

Another aspect of the invention is a method for treating a cancer, tumor or neoplasm, which expresses a metalloproteinase, comprising administering to a subject in need thereof an effective amount of a conjugate according to the invention. The conjugate of the invention preferably contains a cleavable linker that is cleaved by the matrix metalloproteinase(s) predominantly expressed by the tumor or cancer cell. For example, when the cancer, tumor or neoplasm expresses matrix metalloproteinase 2 (MMP2), then the conjugate will contain a cleavable linker that is cleaved by MMP2, when the cancer, tumor or neoplasm expresses matrix metalloproteinase 9 (MMP9), then the conjugate will contain a cleavable linker that is cleaved by MMP9, and when the cancer, tumor or neoplasm expresses matrix metalloproteinase 13 (MMP13), then the conjugate will contain a cleavable linker that is cleaved by MMP13. Depending on the expression of MMPs by a tumor, neoplasm or cancer, the cleavable linker may be cleaved by other matrix metalloproteinases, such as matrix metalloproteinases 1, 3, 7, or 14.

The type of cancer, tumor or neoplasm expressing an MMP may be selected from the group consisting of prostate cancer, squamous cell carcinoma, basal cell carcinoma, melanoma, uterine cancer, glioblastoma, lymphoma, breast cancer, mammary tumor, ovarian cancer, colorectal cancer, gastric cancer, hepatocellular carcinoma, and pancreatic cancer.

### EXAMPLE

Alpha-amantin is first reacted with thionyl chloride in a 1:1 ratio to chemically modified alpha-amanitin by converting aliphatic —OH groups to —Cl groups. Chemical modification of —OH group(s) on the periphery of alpha-amanitin may be favored by steric and electronic effects.

The modified chloride derivative of alpha-amanitin is then reacted with PAMAM (SEQ ID NO: 2) to link the molecules with the loss of HCl.

Free hydroxyl groups of the cell penetrating peptide PLGLAG (SEQ ID NO: 1) were converted to chlorides by reacting with thionyl chloride and the PLGLAG (SEQ ID NO: 1) peptide(s) was linked amantin-PAMAM conjugate by reaction with the 3 free —NH₂ groups to produce a conjugate containing a MMP cleavable linker PLGLAG (SEQ ID NO: 1) between the PAMAM (SEQ ID NO: 2) moiety and the releasable alpha-amanitin peptide.

The conjugate is then purified and mixed with normal saline for in vivo administration.

Nude mice harboring HT-108 tumors (3-8 mm) are obtained from Explora Biosciences and are daily injected intravenously or into a tumor mass with a 1% colloidal dispersion of for two weeks. Positive and negative control groups are respectively injected daily with an equivalent molar amount of unconjugated alpha-amanitin or with an equivalent volume of normal saline.

After three weeks tumor sizes and weights is determined in all groups.

### REFERENCES

1. Kerkelä, E, et al., Matrix metalloproteinases in tumor progression: focus on basal and squamous cell skin cancer. Experimental Dermatology, 2003: p. 109-125.
2. Jiang, T., et al., Tumor imaging by means of proteolytic activation of cell-penetrating peptides. PNAS 101(51)(2004).
3. Mansour, A. M., et al., A New Approach for the Treatment of Malignant Melanoma: Enhanced Antitumor Efficacy of an Albumin-binding Doxorubicin Prodrug That Is Cleaved by Matrix Metalloproteinase 2. CANCER RESEARCH, 2003: p. 4062- 4066.
4. Folgueras, A., et al., Matrix metalloproteinases in cancer: from new functions to improved inhibition strategies. Int. J. Dev. Biol., 2004.
5. Köhrmann, A., et al., Expression of matrix metalloproteinases (MMPs) in primary human breast cancer and breast cancer cell lines: New findings and review of the literature. BMC Cancer, 2009. 9(188).
6. Gimeno-Garcia, A. Z., et al., Up-regulation of gelatinases in the colorectal adenoma-carcinoma sequence. EUROPEAN JOURNAL OF CANCER, 2006: p. 3246-3252.
7. Binder, C., Relaxin enhances in vitro invasiveness of breast cancer cell lines by upregulation of matrix metalloproteases. Molecular Human reproduction, 2002. 8(9): p. 789-796.
8. Hojilla, C. V., Metalloproteinases as common effectors of inflammation and extracellular matrix breakdown in breast cancer. Cancer Research, 2008. 10(205).
10. Trudel, D., et al., Significance of MMP2 Expression in Prostate Cancer: an Immunohistochemical Study. Cancer Res, 2003: p. 8511- 8515.
11. Olson, E., et al., In vivo characterization of activatable cell penetrating peptides for targeting protease activity in cancer. Integr Biol, 2009: p. 382-393.
12. Owen, J. L., et al., Up-Regulation of Matrix Metalloproteinase-9 in T Lymphocytes of Mammary Tumor Bearers: Role of Vascular Endothelial Growth Factor. The Journal of Immunology, 2003: p. 4340-4351.
13. Kashman, A., The expression of Matrix metalloproteinase-2 and cyclooxygenase-2 by immunohistochemistry in patients with colorectal carcinoma. Fac Med Baghdad, 2011. 54(3).
14. Hidalgo, M. Development of Matrix Metalloproteinase Inhibitors in Cancer Therapy. Journal of the National Cancer Institute, 2001. 93(3).
15. Lafleur, M., et al., Upregulation of matrix metalloproteinases (MMPs) in breast cancer xenografts: A major induction of stromal MMP13. Int. J. Cancer, 2005: p. 544-554.
16. Maatta, M., et al., Differential Expression of Matrix Metalloproteinase (MMP)-2, MMP9, and Membrane Type 1-MMP in Hepatocellular and Pancreatic Adenocarcinoma: Implications for Tumor Progression and Clinical Prognosis. 2000. 6: p. 2726-2734.
17. Massimo, L, et al., Uterine cervical carcinoma: Role of matrix metalloproteinases (Review). International Journal of Oncology, 2009: p. 897-903.
18. Wu, M-H, et al., Galectin-1-Mediated Tumor Invasion and Metastasis, Up-Regulated Matrix Metalloproteinase Expression, and Reorganized Actin Cytoskeletons. Mol Cancer Res, 2009: p. 311-318.
19. Mizuma, M., Up-regulated p27Kip1 Reduces Matrix Metalloproteinase-9 and Inhibits Invasion of Human Breast Cancer Cells. Anticancer Research, 2008: p. 2669-2678.
20. Noel, V, et al., Breast cancer progression: insights into multifaceted matrix metalloproteinases. Clin Exp Metastasis, 2007: p. 647-656.
21. Ping Guo, Y. I., et al., Up-Regulation of Angiopoietin-2, Matrix Metalloprotease-2, Membrane Type 1 Metalloprotease, and Laminin 5 gamma 2 Correlates with the Invasiveness of Human Glioma. American Journal of Pathology, 2005. 166(3).
22. Qing-Quan Li, et al., Up-regulation of CD147 and matrix metalloproteinase-2, -9 induced by P-glycoprotein substrates in multidrug resistant breast cancer cells. Cancer Sci, 2007. 98(11): p. 1767-1774.
23. Quyen, T., et al. Surgery with molecular fluorescence imaging using activatable cell-penetrating peptides decreases residual cancer and improves survival. PNAS, 2010. 107(9): p. 4317-4322.
24. Westermark, J, et al. FASEB J. 13, 781-792 (1999). Regulation of matrix metalloproteinase expression in tumor invasion.
25. Rundhaug, J. E., Matrix Metalloproteinases, Angiogenesis, and Cancer. Clinical Cancer Research, 2003. 9: p. 551-554.
(33) 26. Sang-Oh Yoon, et al., Roles of Matrix Metalloproteinases in Tumor Metastasis and Angiogenesis. Journal of Biochemistry and Molecular Biology, 2003. 36: p. 128-137.
27. Pedersen, T. X., et al., Extracellular protease mRNAs are predominantly expressed in the stromal areas of microdissected mouse breast carcinomas. Carcinogenesis, 2005. 27(7): p. 1233-1240.
28. Todd A. Aguilera1, Margaret M. Timmers1, Emilia S. Olson1,2, Tao Jiang1,3, and Roger Y. Tsien1,3, Published as: Integr Biol (Camb). 2009 June; 1(5-6): 371-381.
   Systemic in vivo distribution of activatable cell penetrating peptides is superior to cell penetrating peptides. Integr Biol 2009: p. 371-381.
29. Faulstich, H., et al., Use of conjugates of amatoxin and phallotoxins with macromolecules for cancer and inflammatory therapy.
30. Egeblad M, et al., New functions for the matrix metalloproteinases in cancer progression. Nature Reviews 2002; 2:161-74.
31. Coussens L M, et al., Matrix metalloproteinase inhibitors and cancer: Trials and Tribulations. Science 2002; 295:2387-92.
32. Hua, H., et al., Matrix metalloproteinases in tumorigenesis: an evolving paradigm. Cellular and Molecular Life Sciences, 2011: p. 3853-3868.

1. A conjugate comprising:
a releasable cell alpha-amanitin peptide linked to
a matrix metalloproteinase-cleavable linker that is cleavable by a matrix
a dendrimer or other carrier to which said releasable alpha-amanitine peptide and cleavable linker are conjugated,
wherein the amanitin peptide is releasable from the conjugate when contacted with matrix metalloproteinase that cleaves the cleavable linker.

2. The conjugate of claim 1, wherein releasable alpha-amanitin peptide is alpha-amanitin.

3. The conjugate of claim 1, wherein the cleavable linker is cleavable by matrix metalloproteinase 2 (MMP2).

4. The conjugate of claim 1, wherein the cleavable linker is cleavable by matrix metalloproteinase 9 (MMP9).

5. The conjugate of claim 1, wherein the cleavable linker comprises the amino acid sequence PLGLAG (SEQ ID NO: 1).

6. The conjugate of claim 1, further comprising a moiety that binds to or is taken up by a tumor or cancer cell.

7. The conjugate of claim 1, further comprising a detectable marker.

8. A composition comprising the conjugate of claim 1 and a pharmaceutically acceptable excipient.

9. The composition of claim 8, further comprising at least one agent that increases activity of a matrix metalloproteinase expressed by a tumor, neoplasm or cancer cell.

10. A method for treating a cancer, tumor, or neoplasm which expresses a metalloproteinase, comprising administering to a subject in need thereof an effective amount of the conjugate of claim 1.

11. The method of claim 10, wherein the cancer, tumor or neoplasm expresses matrix metalloproteinase 2 (MMP2) and the cleavable linker is cleaved by MMP2.

12. The method of claim 10, wherein the cancer, tumor or neoplasm expresses matrix metalloproteinase 9 (MMP9) and the cleavable linker is cleaved by MMP9.

13. The method of claim 10, wherein the cancer, tumor or neoplasm is prostate cancer.

14. The method of claim 10, wherein the cancer, tumor or neoplasm is selected from the group consisting of uterine cancer, breast cancer, mammary tumor, and ovarian cancer.

15. The method of claim 10, wherein the cancer, tumor or neoplasm is squamous cell carcinoma, basal cell carcinoma, or melanoma.

16. The method of claim 10, wherein the cancer, tumor or neoplasm is selected from the group consisting of glioblastoma, lymphoma, colorectal cancer, gastric cancer, hepatocellular carcinoma, and pancreatic cancer.
